(19) United States
(12) Patent Application Publication
(10) Pub. No.: US 2006/0241766 A1
(43) Pub. Date: Oct. 26, 2006

(54) Method and apparatus for preventing articulation in an artificial joint

(75) Inventors: Sharonda T. Felton, Cordova, TN (US); Jeff R. Justis, Collierville, TN (US)

Correspondence Address: HAYNES AND BOONE, LLP, 901 MAIN ST, SUITE 3100, DALLAS, TX, 75202, US

(73) Assignee: SDGI Holdings, Inc., Wilmington, DE (US)

(21) Appl. No.: 11/110,375

(22) Filed: Apr. 20, 2005

(51) Int. Cl. A61F 2/44; A61F 2/30

(52) U.S. Cl. 623/17.12; 623/17.13; 623/17.15; 623/23.41

(57) ABSTRACT

An artificial joint is configured for surgical insertion between two bones. The joint includes first and second parts supported for relative movement, and structure that can be selectively used to facilitate relative fixation of the first and second parts in a manner preventing the relative movement thereof. A method involves surgically inserting such a joint between two bones, and completing the surgical procedure with the first and second parts movable relative to each other. A different method relates to a situation where such a joint was previously surgically installed, and involves modifying the joint in situ to fix the first and second parts against relative movement.

### BACKGROUND

Spinal columns have a plurality of vertebrae that are separated by discs. A disc may be displaced or damaged due to trauma or disease, resulting in disruption of the annulus fibrosis, and the eventual protrusion of the nucleus pulposus into the spinal canal. This condition is commonly referred to as a herniated or ruptured disc. The extruded nucleus pulposus may press on the spinal nerve, thereby causing nerve damage, pain, numbness, muscle weakness and/or paralysis. Alternatively, the normal aging process may cause a disc to deteriorate. For example, as a disc ages, it dehydrates and hardens, and this in turn reduces the effective thickness of the disc. As a result, there can be pain, decreased mobility, and/or instability of the spine.

It has become fairly common to surgically remove a damaged or problematic disc, in and to replace it with an artificial disc. One type of artificial disc is designed to secure the adjacent vertebrae against movement with respect to each other, and this is commonly known as fusion of the two vertebrae. When two vertebrae are fused in this manner, the rest of the spinal column provides sufficient movement to accommodate the needs of the patient.

A different type of artificial disc is designed to preserve motion between two vertebrae. This type of disc is designed to operate reliably for many years after it has been surgically implanted in a patient, typically for the natural lifetime of the patient. Nevertheless, in rare situations, problems may eventually develop. For example, even where the artificial disc is still functioning properly, the patient may be subjected to trauma or disease that leads to a physiological condition causing pain, numbness, muscle weakness or the like during the movement permitted by the artificial disc. Alternatively, trauma or long-term wear may cause the artificial disc itself to experience a problem that causes pain or discomfort during the movement permitted by the artificial disc. When one of these types of problems develops, the current solution is to subject the patient to another major surgical procedure, in which the motion preservation disc is surgically removed, and replaced with a new artificial disc. The new disc may be either a motion preservation disc or a fusion disc, depending on the particular circumstances of the patient.

### SUMMARY

One form of the invention involves an artificial joint for surgical insertion between two bones, the joint including: first and second parts supported for relative movement; and structure that can be selectively used to facilitate relative fixation of the first and second parts in a manner preventing the relative movement.

A different form of the invention involves a method of carrying out a surgical procedure that includes: inserting between two bones an artificial joint having first and second parts that are movable relative to each other and that each cooperate with a respective bone, the joint having structure that can be selectively used to facilitate fixation of the first and second parts against relative movement; and completing the surgical procedure with the first and second parts movable relative to each other.

Still another form of the invention relates to a method that involves an artificial joint disposed between two bones and having first and second parts movable relative to each other; wherein the method includes modifying the joint in situ to fix the first and second parts against relative movement.

### BRIEF DESCRIPTION OF THE DRAWINGS

FIG. 1 is a diagrammatic perspective view of an artificial joint that is an intervertebral disc, and that embodies aspects of the present invention.

FIG. 2 is a diagrammatic perspective view, partly in section, showing the disc of FIG. 1 implanted between two vertebrae.

FIG. 3 is a diagrammatic perspective view, partly in section, showing an intervertebral disc that is an alternative embodiment of the intervertebral disc of FIGS. 1 and 2.

FIG. 4 is a central sectional side view of the disc of FIG. 3.

FIG. 5 is a diagrammatic perspective view, partly in section, of an intervertebral disc that is an alternative embodiment of the intervertebral disc of FIGS. 3 and 4.

FIG. 6 is a diagrammatic view similar to FIG. 5, but showing a different operational position.

### DETAILED DESCRIPTION

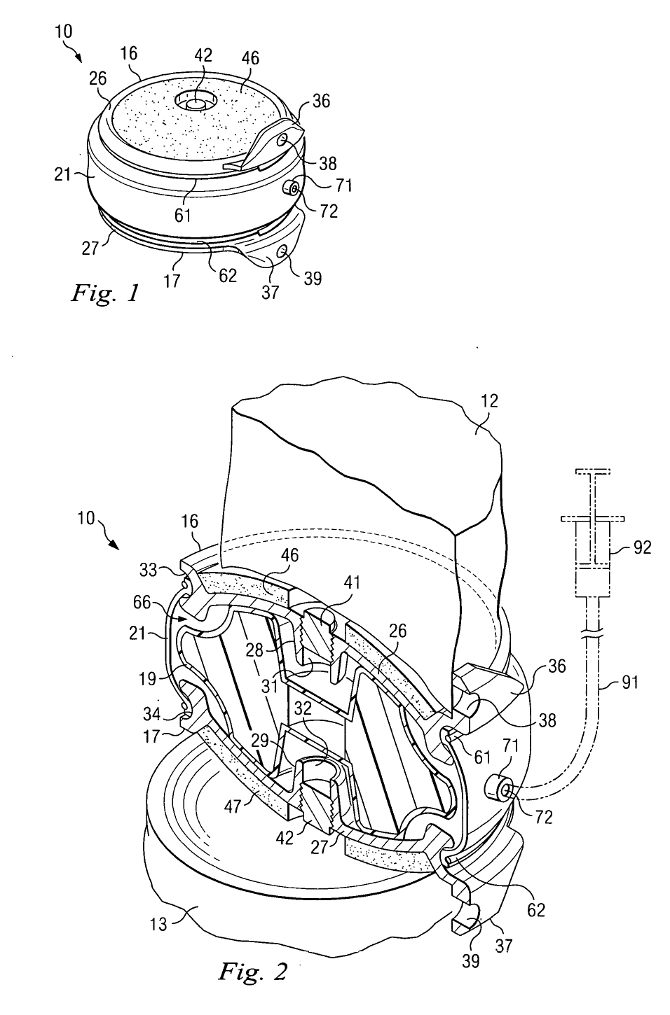

FIG. 1 is a diagrammatic perspective view of an apparatus that is an artificial joint, in particular an intervertebral disc 10. FIG. 2 is a diagrammatic perspective view, partly in section, showing the disc 10 after surgical insertion between two vertebrae 12 and 13. With reference to FIGS. 1 and 2, the disc 10 includes two parts 16 and 17 that are vertically spaced, a central body 19 disposed between the parts 16 and 17, and an annular sheath 21. The sheath 21 encircles the central body 19, and extends vertically between the parts 16 and 17.

The parts 16 and 17 each include a respective shell 26 or 27. The shells 26 and 27 each have a concave inner surface, and a convex outer surface. Further, the shells 26 and 27 each have a central post 28 or 29 that projects vertically toward the other thereof. An opening 31 or 32 extends vertically through each shell 26 or 27, and through the post 28 or 29 thereof. The outer end of each opening 31 and 32 is threaded. The shells 26 and 27 each have a respective annular groove 33 or 34 extending circumferentially around the periphery thereof. The shells 26 and 27 each have an upwardly-extending flange 36 or 37 on a rear side thereof, and a respective opening 38 or 39 extends horizontally through each of the flanges 36 and 37. The parts 16 and 17 also include respective plugs 42 and 43. The plugs 42 and 43 each threadedly engage the threaded outer end of a respective one of the openings 31 and 32. The shells 26 and 27 and the plugs 42 and 43 can each be made from a wide variety of biocompatible materials. In the embodiment of FIGS. 1 and 2 they are made from titanium, but they could alternatively be made from stainless steel, a titanium alloy, a polymeric material such as polyethylene, or any other suitable material.

Each of the parts 16 and 17 has on the convex outer surface thereof a respective coating 46 or 47 that promotes ingrowth of bone material, in order to help fixedly couple the parts 16 and 17 to the bones 12 and 13. In the embodiment of FIGS. 1 and 2, the coatings 46 and 47 are defined by a plurality of sintered beads made of a biocompatible material. In the embodiment of FIGS. 1 and 2 they are made from titanium, but could alternatively be made from stainless steel, a titanium alloy, a polymeric material such as polyethylene, or any other suitable material.

The central body 19 is annular, with a vertical axial opening therethrough. The opposite ends of this opening receive the respective posts 28 and 29, with sufficient clearance to allow relative transverse movement. The central body 19 has convex top and bottom surfaces that each slidably engage the concave inner surface on a respective one of the shells 26 and 27. The central body 19 is resiliently deformable, and has surface regions that are harder then the interior region. This allows the central body 19 to be sufficiently deformable and resilient so that the disc 10 functions to provide resistance to compression and also to provide damping, while still providing adequate surface durability and wear resistance. In addition, the material of the central body is selected so that the surfaces are very lubricious, in order to decrease friction between the central body and each of the rigid shells 26 and 27.

The material used to make the central body 19 is a biocompatible polymeric material that is slightly elastomeric, and that may be coated or impregnated to increase surface hardness, lubricity or both. Coating may be carried out by any suitable technique, such as dip coating, and the coating solution may include one or more polymers. The coating polymer may be the same as or different from the polymer used to form the interior of the central body, and may have a different Durometer hardness than that of the interior material. The coating thickness can be greater than about 1 mil, for example from about 2 mil to about 5 mil. Examples of suitable commercially-available materials include polyurethanes such as polycarbonates and polyethers, including CHRONOTHANE P 75A or P 55D (P-eth-PU aromatic, CT Biomaterials), CHRONOFLEX C 55D, C 65D, C 80A, or C 93A (PC-PU aromatic, CT Biomaterials), ELAST-EON II 80A (Si-PU aromatic, Elastomedic), BIONATE 55D/S or 80A-80A/S (PC-PU aromatic with S-SME, PTG), CARBOSIL-10 90A (PC-Si-PU aromatic, PTG), TECOTHANE TT-1055D or TT-1065D (P-eth-PU aromatic, Thermedics), TECOFLEX EG-93A (P-eth-PU aliphatic, Thermedics), or CARBOTHANE PC 3585A or PC 3555D (PC-PU aliphatic, Thermedics).

The disc 10 includes two retaining rings 61 and 62 that each sealingly hold a respective axial end of the sheath 21 within a respective one of the grooves 33 or 34. An annular chamber 66 is defined within the disc 10, between the sheath 21, the periphery of the central body 19, and the peripheral edges of the shells 26 and 27. In the embodiment of FIGS. 1 and 2, the rings 61 and 62 are made of titanium, but they could alternatively be made of any other suitable biocompatible material, including stainless steel, a titanium alloy, or a synthetic material. The sheath 21 is made from a biocompatible material that is durable and flexible, and that can be slightly elastic. For example, the sheath 21 can be made from a segmented polyurethane having a thickness ranging from about 5 to about 30 mils, and more particularly from about 10 to 11 mils. Examples of suitable commercially-available materials include BIOSPAN-S (aromatic polyetherurethaneurea with surface modified end groups, Polymer Technology Group), CHRONOFLEX AR/LT (aromatic polycarbonate polyurethane with low-tack properties, CardioTech International), CHRONOTHANE B (aromatic polyether polyurethane, CardioTech International), and CARBOTHANE PC (aliphatic polycarbonate polyurethane, Thermedics).

A fitting 71 is mounted on the sheath 21, in angular alignment with the flanges 36 and 37. The fitting 71 extends through the sheath 21, and has a passageway 72 that can provide communication between the annular chamber 66 and the exterior of the disc 10. In the embodiment of FIGS. 1 and 2, the fitting 71 is manufactured with an integral portion that completely obstructs the passageway 72, so as to prevent fluid flow in either direction through the passageway 72. As discussed in more detail later, the obstruction can be selectively punctured at a later point in time, in order to allow fluid flow. As an alternative to the obstruction, the fitting 71 could have a valve to control fluid flow through the passageway 72, such as a simple spring-biased ball valve of a known type. The fitting 71 can be made from a wide variety of materials that are biocompatible. In the embodiment of FIGS. 1 and 2 the fitting is made from a polymeric material such as polyethylene, so that the integral obstruction in the passageway 72 can be punctured without difficulty. However, the fitting 71 could be made from any other suitable material. If it included a valve rather than the integral obstruction, then it could be made from materials such as titanium, stainless steel, or a titanium alloy.

Following manufacture of the disc 10, the disc 10 is surgically inserted in a known manner between two vertebrae, such as the vertebrae shown at 12 and 13 in FIG. 2. Not-illustrated screws can optionally be inserted through the openings 38 and 39 in the flanges 36 and 37, in order to engage the bones 12 and 13 and thus securely hold the disc 10 in place. Over time, and as mentioned above, bone growth will occur into the sintered coatings 46 and 47, thereby further securing the disc 10 to the bones 12 and 13.

After surgical insertion of the disc 10, and after recovery of the patient, the disc will facilitate a degree of relative movement between the bones 12 and 13. In particular, the shells 26 and 27 can each carry out limited lateral sliding movement relative to the central body 19. Since the cooperating surfaces on the central body 19 arid the shells 26 and 27 are curved, the relative movement will effectively be limited pivotal movement about any of various horizontal axes. In addition, the inherent resilience of the central body 19 will allow a limited degree of vertical compression that permits movement of the shells toward each other, and also a limited degree of relative rocking movement of the shells that is effectively limited pivotal movement about horizontal axes.

In rare cases, it is possible that a problem may develop over time. For example, even where the disc 10 is still functioning properly, the patient may experience trauma or disease that leads to a physiological condition causing pain, numbness, muscle weakness or the like during the relative vertebral movement permitted by the disc 10. As another example, trauma or long-term wear may cause the disc 10 itself to experience a problem that causes pain or discomfort to the patient during the movement permitted by the disc. In either case, the standard solution with pre-existing artificial discs is to subject the patient to a further major surgery in order to replace the artificial disc with a different artificial disc. In contrast, the disc 10 allows a different approach.

More specifically, in a relatively minor surgery, a small incision is made in the skin and muscle of the patient, order to allow access to the fitting 71. The obstruction within the passageway 72 is punctured with a sharp and sterile object, in order to permit fluid flow through the passageway 72. One end of a tube 91 is then coupled to the fitting 71 in any suitable manner, so that the passageway 72 is in fluid communication with the passageway that extends through the tube 91. A syringe 92 or other suitable device is then used to inject a fluid material through the tube 91 and fitting 71, in order to fill the chamber 66 with the material. The material then cures or hardens, preferably in a relatively short period of time. Since this material engages the entire peripheral edge of each of the shells 26 and 27, the shells 26 and 27 will become fixed against relative movement when the material hardens. Consequently, the disc 10 will be converted from one operational mode in which the shells 26 and 27 are capable of relative movement to a different operational mode in which the shells 26 and 27 are fixed against any relative movement. The material injected into the chamber 66 is a biocompatible material. In the embodiment of FIGS. 1 and 2, the material is a known epoxy, where two components are mixed together in a fluid state and then injected into the chamber 66, where the mixture chemically hardens. The material could alternatively be any other suitable material, including any of a number of known cements that are initially fluid but then harden.

After the material has been injected, the tube 91 is detached from the fitting 71, and the opening 72 is closed. For example, a small plug may be force-fit into the opening 72. Alternatively, the opening 72 could be closed in any other suitable manner. The small incision made through the skin and muscle of the patient is then sutured or stapled. If necessary, the patient is kept immobilized until the material in the chamber 66 has had time to harden. However, in the embodiment of FIGS. 1 and 2, the material hardens in a relatively short period of time, so that it is fully hardened by the time the surgeon finishes closing the incision and the patient is released to the recovery room. This is a minimally invasive procedure that can be performed on an outpatient basis, and permits the patient to be up and around in a day or two, as opposed to the long recovery time needed for a major surgery in which an artificial disc is removed and replaced with another.

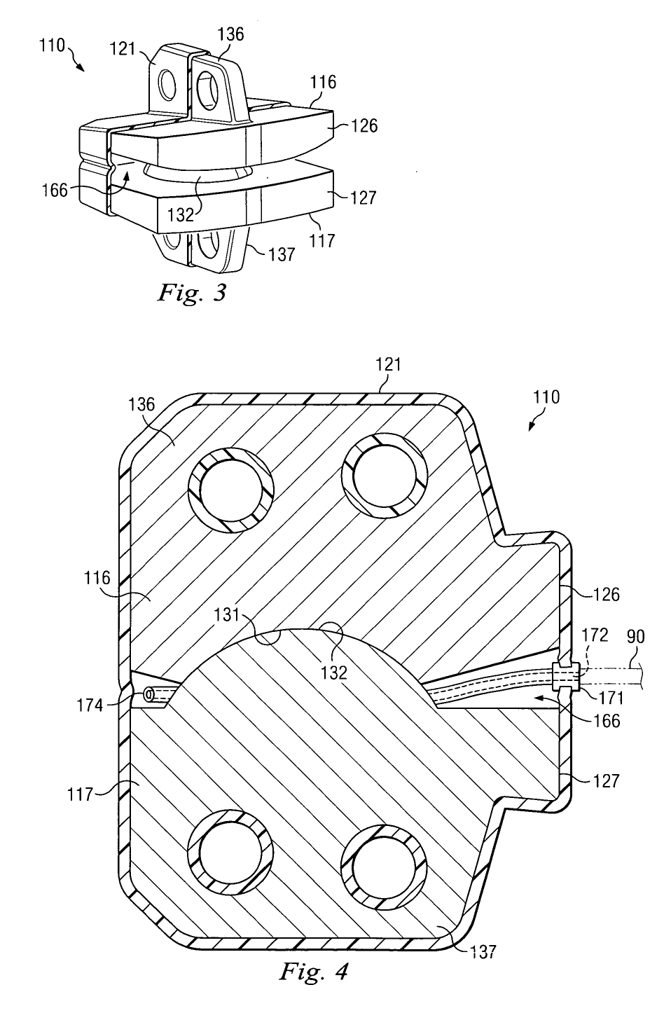

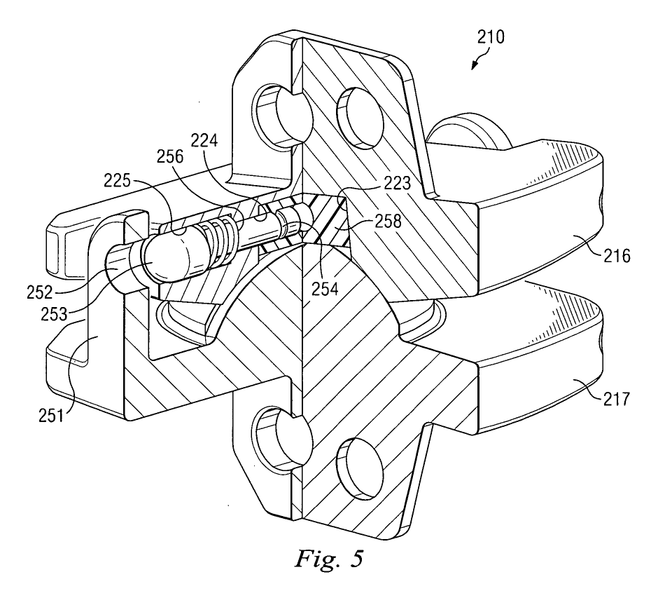

FIG. 3 is a diagrammatic perspective view, partly in section, showing a disc 110 that is an alternative embodiment of the disc 10 of FIGS. 1 and 2. The disc 110 includes two parts 116 and 117, and a sheath 121 that envelopes the parts 116 and 117. Approximately half of the sheath 121 has been removed in FIG. 3, so that the parts 116 and 117 can be seen. FIG. 4 is a central sectional side view of the disc 110 of FIG. 3.

The parts 116 and 117 each include a respective plate-like center portion 126 or 127. The center portion 126 has in the underside thereof an approximately hemispherical recess with a concave surface 131. The center portion 127 has on an upper side thereof an approximately hemispherical projection with a convex surface 132. The surfaces 131 and 132 slidably engage each other, to facilitate approximately pivotal movement of the parts 116 and 117 with respect to each other.

The part 116 has on the upper side of its center portion 126 an upwardly-extending projection or keel 136. Similarly, the part 117 has on the lower side of its center portion 127 a downwardly-extending projection or keel 137. The projections 136 and 137 each have a pair of transverse openings extending therethrough. Before the disc 110 is inserted between two vertebrae, the surgeon creates a recess in each vertebra. Then, when the disc 110 is surgically implanted, the projections 136 and 137 are each received in one of those recesses. This helps to anchor the disc 110 in the proper position. Further, as bone growth occurs over time, there will be bone growth into the transverse openings through the projections 136 and 137, thereby helping to anchor the disc 110 in place. The parts 116 and 117 can be made from a wide variety of biocompatible materials. In the embodiment of FIGS. 3 and 4, the parts 116 and 117 are made from a cobalt-chrome-molybdenum metallic alloy (such as ASTM F799 or F-75). The parts 116 and 117 could alternatively be made from stainless steel, titanium, a titanium alloy, a polymeric material such as polyethylene, or any other suitable material.

The sheath 121 is made of a biocompatible material that is durable and flexible, and that may be slightly elastic. For example, the sheath 121 can be made from materials of the type discussed above in association with the sheath 21 of FIGS. 1-2. The sheath 121 may optionally be made from a material that promotes bone growth. Also, to facilitate bone growth, the top and bottom portions of the sheath 121 can be roughened. Alternatively, the top and bottom portions of the sheath 121 may optionally be coated with a known type of material that promotes bone growth. A variety of bone-growth promoting substances are known in the art. One example is a hydroxyapatite coating formed of calcium phosphate.

As best seen in FIG. 4, an annular chamber 166 is present within the sheath 121, and extends around the hemispherical projection having surface 132, between the peripheral edges of the center portions 126 and 127 of the parts 116 and 117. As shown in FIG. 4, a fitting 171 is mounted in an opening through the sheath 121, on a rear side of the disc 110. The fitting 171 is similar to the fitting 71 that was discussed above in association with FIGS. 1 and 2, and has a passageway 172 extending therethrough. The fitting 171 initially includes an obstruction or valve within the passageway 172, in the same manner as the fitting 71.

A tube 174 is provided within the chamber 166, and has one end fixedly secured to the inner side of the fitting 171. The opening through the tube 174 communicates with the passageway 172, and effectively serves as an extension of the passageway 172. The other end of the tube 174 is positioned on a side of the chamber 166 that is remote from the fitting 171. Although FIG. 4 shows only a single tube 174, it would alternatively be possible to have a plurality of tubes that are all coupled to the fitting 171, and that each extend from the fitting 171 to a respective different location within the chamber 166.

When the disc 110 is surgically implanted in a patient, the parts 116 and 117 are initially capable of relative movement, due to the sliding engagement of the surfaces 131 and 132. If necessary, at a later time, a material can be injected into the chamber 166 in a fluid state, through the fitting 171 and the tube 174. The material then hardens within the chamber 166. The engagement of this hardened material with the peripheral surfaces of the parts 116 and 117 serves to fix the parts 116 and 117 against relative movement. The injection of this material is carried out in a minor surgical procedure that is similar to the procedure already described above in association with the embodiment of FIGS. 1-2. Accordingly, to avoid redundancy, the surgical procedure is not described again here.

In a not-illustrated variation of the embodiment of FIGS. 1 and 2, a lubricant is provided within the disc 10 at the time it is initially manufactured. In particular, after the disc 10 has been substantially fully assembled, and after it has been sterilized, one of the plugs 41 and 42 is installed in one of the openings 31 and 32, and then a lubricant is introduced through the other of the openings 31 and 32. The lubricant may be any suitable material,. such as saline, hyaluronic acid, mineral oil, or the like. The other of the plugs 41 and 42 is then installed in the other opening.

Later, when it becomes necessary to introduce a material such as cement into the chamber 66, there will be a need to remove most or all of the lubricant that is in the chamber 66. In that event, the fitting 71 and the tube 90 may each have two passageways, one of which carries the material that is being injecting into the chamber, and the other of which allows the lubricant to escape from the chamber. With respect to the passageway for the material being injected, the disc 10 would include a tube similar to that shown at 174 in FIG. 4, so that the injected material is introduced on a side of the chamber 66 remote from the fitting 71. As the injected material progressively fills the chamber 66, it forces the lubricant to progressively flow to the fitting 71, and then out through the extra passageway in the fitting 71 and tube 90.

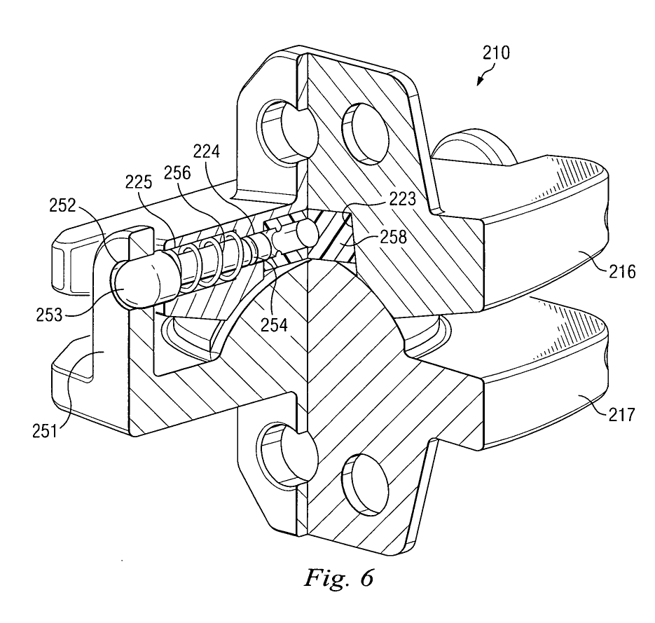

FIG. 5 is a diagrammatic perspective view, partly in section, of an intervertebral disc 210 that is an alternative embodiment of the intervertebral disc 110 of FIGS. 3 and 4. FIG. 6 is a diagrammatic view similar to FIG. 5, but showing a different operational position of the disc 210. The disc 210 includes two parts 216 and 217 that are generally similar to the parts 116 and 117 described above in association with FIGS. 3 and 4, except for the differences discussed below.

The part 216 has an approximately rectangular recess 223 in the center thereof. A cylindrical hole extends horizontally through the part 216, and has two portions 224 and 225 of different diameter. The portion 224 is of smaller diameter than the portion 225, and communicates at its inner end with the recess 223. The outer end of the portion 225 opens through an exterior surface of the part 216. The part 217 has an upwardly projecting post 251, and an opening 252 extends horizontally through the upper end of the post 251.

The disc 210 includes a pin 253 that is axially slidably disposed within the opening 224 and 225 in the part 216. The pin 253 has an annular groove near its inner end. A coil spring 256 encircles the pin 253, and resiliently urges the pin 253 to move axially outwardly. The recess 223 in the part 216 is filled with a material 258. As shown in FIG. 5, the material 258 engages the groove 254 in the pin 253, and prevents the pin 253 from being moved axially outwardly by the spring 256.

In the embodiment of FIGS. 5 and 6, the material 258 is a material that is commercially available under the tradename TERFENOL-D from Etrema Products, Inc. of Ames Iowa. Normally, the material 258 is relatively rigid. However, when subjected to an appropriate field of electromagnetic energy, the material 258 undergoes a shape change. This permits the spring 256 to move the pin 253 outwardly to the position shown in FIG. 6, where the outer end of the pin 253 engages the opening 252 in the post 251 on the part 217. This mechanically locks the parts 216 and 217 against any relative movement, even after the electromagnetic field is removed and the material 258 returns to its original shape. The electromagnetic field can be applied to the material 258 without any need to make any incision in the patient.

Instead of the TERFENOL-D product discussed above, the material 258 could alternatively be any other suitable material that. can transition between two states, such as hard and soft states. For example, the material 258 could be a polyethylene material having an electrically conductive part embedded in it. When subjected to a rapidly varying magnetic field, an electric current is induced in the electrically conductive part and causes it to heat up, which in turn heats the polyethylene in order to soften it sufficiently so that the pin 253 is released.

Although selected exemplary embodiments have been disclosed above in detail, many modifications and variations are possible. For example, it would alternatively be possible to provide a disc having a cam or other mechanical element that can be selectively manually moved between two positions in which it respectively permits and obstructs relative movement of two parts. As another alternative, a mechanical element that is not initially present in the disc could be selectively manually inserted in order to obstruct relative movement of two parts. Persons skilled in the art will readily appreciate that many other modifications and variations are possible without departing from the spirit and scope of the invention, as defined by the claims that follow.

The foregoing description uses spatial references such as “horizontal,” “vertical,” “top,” “upper,” “lower,” “bottom,” “left,” and “right”, in relation to orientations that are shown in the drawings. These spatial references are used for purposes of convenience, and are not intended to limit the scope of protection provided by the claims that follow. In the claims, means-plus-function clauses are intended to cover the structures described herein as performing the recited function and not only structural equivalents, but also equivalent structures.

What is claimed is:

1. An apparatus comprising an artificial joint for surgical insertion between two bones, the joint including:
first and second parts supported for relative movement; and
structure that can be selectively used to facilitate relative fixation of the first and second parts in a manner preventing the relative movement.

2. An apparatus according to claim 1, wherein the artificial joint is an artificial disc adapted for surgical insertion between two adjacent vertebrae.

3. An apparatus according to claim 1, wherein the structure includes a chamber, and a passageway that communicates with the chamber.

4. An apparatus according to claim 3, including a material that can be introduced into the chamber through the passageway in a substantially fluid state, and that subsequently hardens within the chamber to effect the relative fixation of the first and second parts.

5. An apparatus according to claim 4, wherein the material engages each of the first and second parts when disposed within the chamber.

6. An apparatus according to claim 3, wherein the structure includes an annular sheath that extends between and is coupled to each of the first and second parts, the chamber being located between the first and second parts and within the sheath.

7. An apparatus according to claim 6, wherein the structure includes a fitting that is supported on the sheath and that has having the passageway extending therethrough.

8. An apparatus according to claim 6, including a resilient member disposed within the sheath and engaging each of the first and second parts in a manner facilitating the relative movement thereof, the chamber being annular and extending around the resilient member.

9. An apparatus according to claim 3, wherein the structure includes an envelope that has the first and second parts and the chamber therein.

10. An apparatus according to claim 9, wherein the structure includes a fitting that is supported on the sheath and that has the passageway extending therethrough.

11. An apparatus according to claim 10, wherein the structure includes a tube that extends from the fitting to a location within the chamber and spaced from the fitting, the tube having a portion of the passageway extending therethrough.

12. An apparatus according to claim 9, wherein the first and second parts have respective first and second surfaces thereon that slidably engage each other to facilitate the relative movement of the first and second parts.

13. An apparatus according to claim 12, wherein the chamber includes a portion that is annular, that is disposed between the first and second parts, and that extends around the first and second surfaces.

14. A method comprising carrying out a surgical procedure that includes:
surgically inserting between two bones an artificial joint having first and second parts that are movable relative to each other and that each cooperate with a respective bone, the joint having structure that can be selectively used to facilitate fixation of the first and second parts against relative movement; and
completing the surgical procedure with the first and second parts movable relative to each other.

15. A method according to claim 14,
wherein the artificial joint is an artificial disc; and
wherein the surgically inserting includes inserting the artificial disc between two adjacent vertebrae.

16. A method involving an artificial joint that is disposed between two bones and that has first and second parts movable relative to each other; the method comprising:
modifying the joint in situ to fix the first and second parts against relative movement.

17. A method according to claim 16, including surgically creating access to the artificial joint before the modifying thereof.

18. A method according to claim 16,
wherein the artificial joint is an artificial disc and the bones are adjacent vertebrae; and
wherein the surgically creating access includes creating access to the region of the disc located between the vertebrae.

19. A method according to claim 16, wherein the joint includes a chamber; and wherein the modifying includes introducing into the chamber a material that is in a substantially fluid state, and that subsequently hardens within the chamber to effect the fixation of the first and second parts against relative movement.

20. An apparatus comprising an artificial joint for surgical insertion between two bones, the joint including:
first and second parts;
first means cooperable with the first and second parts for facilitating relative movement thereof; and
second means that can be selectively utilized for facilitating fixation of the first and second parts against relative movement.

21. An apparatus according to claim 20, wherein the second means includes means defining a chamber, and means defining a passageway that communicates with the chamber.

22. An apparatus according to claim 21, wherein the second means includes a material that can be introduced into the chamber through the passageway in a substantially fluid state, and that subsequently hardens within the chamber to effect the relative fixation of the first and second parts.

23. An apparatus according to claim 21, wherein the second means includes an annular sheath that extends between and is coupled to each of the first and second parts, the chamber being located between the first and second parts and within the sheath.

24. An apparatus according to claim 21, wherein the second means includes an envelope that has the first and second parts and the chamber therein.
